(19) United States
(12) Patent Application Publication
(10) Pub. No.: US 2009/0246262 A1
(43) Pub. Date: Oct. 1, 2009

(54) EASILY APPLIED FIELD DRESSING FOR WOUNDS

(75) Inventor: Charles W. Kerber, La Mesa, CA (US)

Correspondence Address: KNOBBE MARTENS OLSON & BEAR LLP, 2040 MAIN STREET, FOURTEENTH FLOOR, IRVINE, CA, 92614, US

(73) Assignee: Valor Medical, Inc., San Diego, CA (US)

(21) Appl. No.: 12/411,780

(22) Filed: Mar. 26, 2009

Related U.S. Application Data
(60) Provisional application No. 61/040,560, filed on Mar. 28, 2008.

(51) Int. Cl. A61K 9/70; A61K 31/785; A61P 17/02

(52) U.S. Cl. 424/445; 424/78.06

(57) ABSTRACT

A method for treating wounds by adhering a wound dressing to a wound with a polymerizable alkyl cyanoacrylate composition, such that the wound dressing and cyanoacrylate composition seal the wound to inhibit bleeding and infection. The dressing may be in the form of a mitten. Also disclosed is injection of polymerizable cyanoacrylate into puncture wounds to seal the wounds and inhibit bleeding.

### CROSS-REFERENCE TO RELATED APPLICATIONS

This application claims the benefit under 35 U.S.C. §119(e) of U.S. Provisional Application No. 61/040,560, filed Mar. 28, 2008, which is incorporated herein by reference in its entirety.

### BACKGROUND OF THE INVENTION

1. Field of the Invention

This invention is directed to field dressings for wounds that can be easily applied by unskilled personnel, at least partially sealing the wound by use of an alkyl cyanoacrylate adhesive composition.

2. Description of the Related Art

Cyanoacrylates are well known in the art and can be represented by formula I:

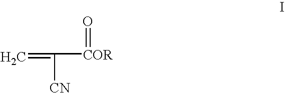

wherein R is an alkyl or other suitable substituent. Such cyanoacrylates are disclosed in U.S. Pat. Nos. 3,527,224; 3,591,676; 3,667,472; 3,995,641; 4,035,334; and 4,650,826. Typically, when applied onto living tissue, the R substituent is alkyl of from 2 to 10 carbon atoms.

Alkyl alpha cyanoacrylates are a homologous series of organic molecules which polymerize and can adhere to moist living tissues. The methyl homolog has been used in homeostasis and non-suture closure since 1960, but its histoxicity severely limited its clinical usefulness. The synthesis of longer alkyl chain homologs and the evaluation of these in various animal species have shown that the histoxicity of cyanoacrylates could be diminished without sacrificing their hemostatic and tissue bonding properties.

Polymerization speed is another function of chain length. It has been reported that homologs with six or more carbon atoms on the alkyl chain polymerize almost immediately upon contact with moist tissues. The n-butyl and isobutyl monomers require from four to 15 seconds, while the methyl homolog remains as a monomer for 30 to 55 seconds. Rapid polymerization allows the material to set in flowing blood without passing through small channels into venous structures. The ability to wet and spread easily over the surface of an anti-coagulated blood film is common to homologs with alkyl chains containing four or more carbon atoms. The ethyl and propyl derivatives wet and spread poorly, and the methyl not at all.

Cyanoacrylate monomers were first used in military medicine during the early parts of the Vietnam war, and have been credited with saving numerous lives (Matsumoto, T., Tissue Adhesives in Surgery. 1972, New York: Medical Examination Publishing Company. 508). They were spread topically on actively bleeding parenchymal organs (such as liver), and rapidly stopped most hemorrhage.

Those early monomers possessed two unfavorable characteristics which led to the abandonment of the surgical use of this chemical. First, the monomers used were histotoxic because of their short side chains (Eastman 910, a single carbon cyanoacrylate, and ethyl, a two carbon cyanoacrylate were the monomers available at that time). Second, the pure polymer was grasslike, unyielding to further surgical manipulation, and did not tolerate the normal motion found in the body.

More than 10 years passed before an effective modification of the basic character of polymerized cyanoacrylates was discovered. Cromwell and Kerber (Cromwell, L. D. and C. W. Kerber, “Modification of Cyanoacrylate for Therapeutic Embolization: Preliminary Experience,” AJR Am. J. Roentgenol, 1979. 132(5): p. 799-801) added an oily radio-opaque material (ethiodol® or pantopaque), which slowed polymerization to a more controllable rate and softened the resulting polymer. That polymer had the ultimate consistency of dry cheese—but was still not flexible.

It took an additional 15 years before cyanoacrylate based devices were approved by the FDA and made available for general medical (almost always intra-arterial) use. TruFill, marketed by the Cordis Corp., was the original Cromwell formulation—butyl cyanoacrylate plus ethiodol®. Also recently, an eight carbon monomer, Dermabond, has been approved for topical use—for closure of superficial cuts (T. B. Bruns and J. M. Worthington, “Using Tissue Adhesive for Wound Repair: A Practical Guide to Dermabond,” Am. Fam. Physician 2000, 61: p. 1383-1388). The Dermabond disadvantage is that, if allowed to enter the wound, the polymer actually interferes with and delays wound healing.

It is known that alkyl cyanoacrylate compositions can be employed in surgical environments as suture replacements or hemostats. When employed in these environments, the parameters of the alkyl cyanoacrylate composition for such applications often diverge from the compositional parameters for topical application. For example, unlike topical application over large areas of skin, surgical application of the alkyl cyanoacrylate composition is typically applied to a small total surface area inside the human body and, accordingly, parameters such as viscosity, curing time, flexibility, durability, etc., are often different for this application as opposed to the parameters required for topical application.

U.S. Pat. No. 3,995,641 discusses the novelty of modified cyanoacrylates, namely, carbalkoxyalkyl cyanoacrylates. The patent disclose their usefulness as a tissue adhesive in surgical applications. The presumed superiority of these products was attributable to the rapid hydrolytic decay and concurrent low degree of histotoxicity.

The prior art methods and compositions have been able to achieve a synthesis of the alkyl cyanoacrylate at economic levels for applications in the medical field, although improbable for uses in commercial applications due to reaction. A number of methods have been attempted to improve yields. Tseng et al., Biomaterials, 1990, 11, 73-79. The variables looked at included: azeotropes, temperature and formaldehyde/cyanoacetate ratio. Other methods have also included assessment of different catalysts for the condensation reaction. Regardless of the methods tried, yields become increasingly smaller as the cyanoacetate pendent group becomes larger.

There exists a continuing unmet need for methods and products useful for treating battlefield wounds and other significant trauma. This disclosure illustrates the use of cyanoacrylates to meet those needs.

### SUMMARY OF THE INVENTION

The present disclosure includes methods and products for treating trauma wounds with alkyl cyanoacrylate compositions suitable for directly injecting into the wound and/or as a topical sealant in combination with a field wound dressing. The alkyl cyanoacrylate monomer is contacted with the wound at which time it can cure to form a polycyanoacrylate resin. In one embodiment, the polycyanoacrylate resin can completely or substantially seal the wound until further treatment is available. For example, the wound may be a puncture type wound, a bullet wound, a laceration, an abrasion, or any other type of wound that is desirably closed, sealed, or covered to reduce bleeding or reduce the risk of infection.

In some embodiments, the alkyl cyanoacrylate compositions can provide an almost immediate cellular level mechanical barrier to the continued loss of tissue fluids and blood, and a barrier to continued contamination. Further, in some embodiments the alkyl cyanoacrylate compositions can be applied to the wound of a wounded individual by someone with no medical training. For example, military personnel could apply the alkyl cyanoacrylate compositions to wounded colleagues, or to themselves. Similarly, emergency response workers could use these materials at the scene of an accident or disaster to treat inured individuals until more sophisticated medical care is available.

In some embodiments, the alkyl cyanoacrylate compositions can stop bleeding within a battlefield-appropriate time with little manual pressure; for example, within one or two minutes or less, and sometimes within as little as about 10 seconds to about 15 seconds. In some embodiments, the alkyl cyanoacrylate compositions can be used in conjunction with more traditional means of hemorrhage control, for example, tourniquets.

In some embodiments, the type of wound to be treated with the alkyl cyanoacrylate compositions can be a puncture type wound, such as, for example, a puncture, laceration, abrasion, or bullet or shrapnel wound. A syringe may be used to inject an alkyl cyanoacrylate monomer composition into the wound, followed by withdrawal of the syringe. The alkyl cyanoacrylate monomers can then polymerize to form a polycyanoacrylate thermoplastic resin that seals the wound to prevent or lessen further bleeding or oozing of tissue fluids.

In some embodiments, the puncture type wound that has optionally been sealed with polycyanoacrylate resin by injection of alkyl cyanoacrylate monomers into the wound can be externally sealed. For example, the wound can be externally sealed by applying an alkyl cyanoacrylate composition to a dressing (such as a mitten or a film), and then applying the dressing over the wound. The dressing can be removed at a later time when the wounded individual is moved to a treatment facility to undergo surgery. Depending on the dressing type, it can be peeled off, or if necessary can be removed using an appropriate solvent, such as acetone.

In some embodiments, the type of wound to be treated with the alkyl cyanoacrylate compositions can be a laceration or other open type wound. For example, an individual or a medical aid personnel can treat the wound by utilizing a system comprising a topically-applied source of an alkyl cyanoacrylate composition and a surgical dressing, which in one embodiment may advantageously be in the form of a mitten. After unwrapping the pump spray and mitten (or plastic film or other dressing), the individual or medical aid personnel can apply the alkyl cyanoacrylate monomer composition to the dressing or to the wound and area surrounding the wound. In a preferred embodiment, the cyanoacrylate is applied to the dressing. Spray application is particularly preferred. The individual or medical aid personnel can then immediately press the dressing onto the wound, holding it in place a sufficient time for adhesion to occur. The alkyl cyanoacrylate composition can preferably adhere the dressing within about 20-30 seconds, or even more quickly. In some aspects, the dressing can be held in place as long as needed to form a barrier over the wound.

In some embodiments, an open type wound can be bleeding excessively and the alkyl cyanoacrylate monomer can be sprayed over and around the wound to form a polycyanoacrylate thermoplastic resin that seals the wound completely or substantially preventing further bleeding. In some embodiments, a dressing such as a mitten or plastic film can be applied, covering the wound before the alkyl cyanoacrylate monomer polymerizes, wherein upon polymerization the mitten or plastic film and polycyanoacrylate thermoplastic resin seal the wound from further bleeding. The wounded individual can then be transported to treatment facilities for further attention.

### BRIEF DESCRIPTION OF THE DRAWINGS

FIG. 1 is a drawing showing a wound dressing wherein the wound dressing is a mitten that can be used to seal a wound.

FIG. 2 is a drawing showing a wound dressing wherein the wound dressing includes a plastic film that can be used to seal a wound.

FIG. 3 is a drawing showing a wound dressing wherein the wound dressing has one side that is a plastic film and the other side has a surface that does not stick to plastic film.

FIG. 4 is a drawing showing a wound dressing that is rolled into a tube.

### DETAILED DESCRIPTION OF THE PREFERRED EMBODIMENT

This invention relates to treating trauma wounds and other wounds or bleeding with alkyl cyanoacrylate monomer compositions suitable for directly injecting into a wound and/or as a topical sealant in combination with a field wound dressing. The alkyl cyanoacrylate monomer can cure to form a polycyanoacrylate thermoplastic resin that seals the wound from further bleeding or other loss of body fluids.

In areas where access to medical treatment facilities are not readably available, controlling bleeding and sealing a wound in an individual in the geographic area where the person was wounded can be advantageous. For example, military personnel wounded on the battlefield or wounded during the performance of military duties may not have ready access to medical treatment. Emergency response personnel also have need of a ready means to control or stop bleeding in an injured individual until the individual can be moved to an appropriate treatment facility.

Some embodiments relate to a method for treating and/or protecting a puncture type trauma wound. In some embodiments, the method for treating the puncture type trauma wound can include inserting a cannula connected to a syringe containing alkyl cyanoacrylate in monomeric (or oligomeric) form into to the wound and injecting alkyl cyanoacrylate in monomeric form into the wound, wherein upon contact with the aqueous in vivo environment, the alkyl cyanoacrylate monomer polymerizes. The resulting formation of polycyanoacrylate effectively slows or stops the bleeding. In some embodiments, the alkyl cyanoacrylate monomer can be dispensed from a tube. For example, the tube can be a metal tube, a plastic tube, or the like. In some embodiments, the alkyl cyanoacrylate monomer can be dispensed from a syringe. For example, the syringe can be a single use syringe. Preferably, the container of cyanoacrylate is opaque or otherwise protected from light.

In some aspects, the alkyl cyanoacrylate composition can slow or stop seepage of body fluids in addition to slowing or stopping bleeding. For example, the alkyl cyanoacrylate monomer can rapidly polymerize to form a polycyanoacrylate thermoplastic resin that completely or nearly completely seals the wound. In some embodiments, the polycyanoacrylate thermoplastic resin can prevent from about 60% to about 100% of bleeding and/or the seepage of body fluids from a wound. In a preferred embodiment, the polycyanoacrylate thermoplastic resin can prevent from about 90% to about 100% of bleeding and/or the seepage of body fluids in a wound. In a more preferred embodiment, the polycyanoacrylate thermoplastic resin can prevent almost 100% of bleeding or the seepage of body fluids from a wound.

In some embodiments, an additional step of sealing a trauma wound can be useful to prevent bleeding and slow or stop seepage of body fluids. For example, subsequent to or instead of injecting alkyl cyanoacrylate monomer into a puncture-type trauma wound, alkyl cyanoacrylate monomer can be sprayed over and around the wound and then a surgical dressing (optionally in the form of a mitten to allow one-handed self-administration) can be immediately pressed onto the wound. The dressing can be held in place for a long as needed to substantially or completely seal the wound. Upon polymerization of the alkyl cyanoacrylate monomer, the mitten or plastic wound dressing can be bonded to and around the puncture type trauma wound.

In some embodiments, the method of treating the wound can comprise: injecting alkyl cyanoacrylate monomer into the puncture type trauma wound, spraying alkyl cyanoacrylate monomer on or around wound, immediately applying the mitten or plastic wound dressing to the puncture type trauma wound, and continuing to apply pressure until the mitten or plastic wound dressing adheres to the puncture type trauma wound.

Wound dressings disclosed herein can have one or more of the following desirable characteristics:

(1) The dressing material includes a liquid, that can penetrate into the crevasses and topology of a wound and the surrounding skin;

(2) The dressing material blocks vessels and stops bleeding almost immediately;

(3) The dressing system is easy to use, and can be applied by relatively untrained personnel to others or to themselves;

(4) The material is bactericidal;

(5) The material works with other clotting agents, and does not interfere with their action; and

(6) The wound dressing can be readily removed by a surgeon or other professional in a tertiary care environment, without inhibiting or delaying care.

Ideally, for treating open wounds, the alkyl cyanoacrylate compositions should meet as many of the following criteria as possible:

(1) the alkyl cyanoacrylate composition is advantageously stable against premature polymerization. Such formulations would therefore be suitable for storage in applicators for single use applications and in spray applicators where unintended polymerization can clog the spray mechanism;

(2) the alkyl cyanoacrylate composition is preferably of suitable viscosity for desired application into the wound and/or onto the skin;

(3) once applied to the skin, the alkyl cyanoacrylate preferably polymerizes quickly, to form a rapid seal with a wound dressing, and can advantageously cure within from about four seconds to about 2 minutes after application;

(4) the resulting cured polymer advantageously has sufficient flexibility such that the integrity of the polymer coating is not compromised by, for example, cracking, etc; and

(5) is preferably non-toxic when injected into a wound applied to the skin or a wound surface.

### Wound Dressings and Methods

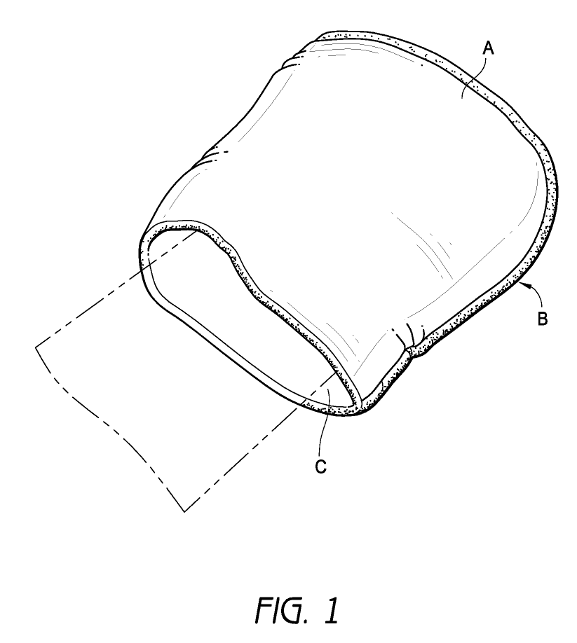

One embodiment of a wound dressing in the form of a mitten is illustrated in FIG. 1. Various sizes of mittens are contemplated; e.g., the mitten can cover the hand up to the wrist, or just the fingers, or the fingers and palm. In a preferred embodiment, the mitten can have at least one surface that is a plastic or other polymer, preferably a film, (FIG. 1, A) so that when alkyl cyanoacrylate monomer is applied and the mitten is pressed against a wound, the plastic adheres to the wound due to polymerization of the alkyl cyanoacrylate monomer. This reduces or eliminates bleeding or oozing of bodily fluids. In a further preferred embodiment, the top and bottom half of the mitten can be separated from each other after adhering to the wound to leave only the half of the mitten sealed to the wound on the patient. For example, the mitten can have a perforated edge (FIG. 1, B) that can be torn to leave a bottom half of the mitten adhered to and around the wound. In some aspects, the half of the mitten sealed over the wound can be left intact until the patient is moved to treatment facilities where it can be removed, for example, by peeling it off, by debridement, or through use of a solvent.

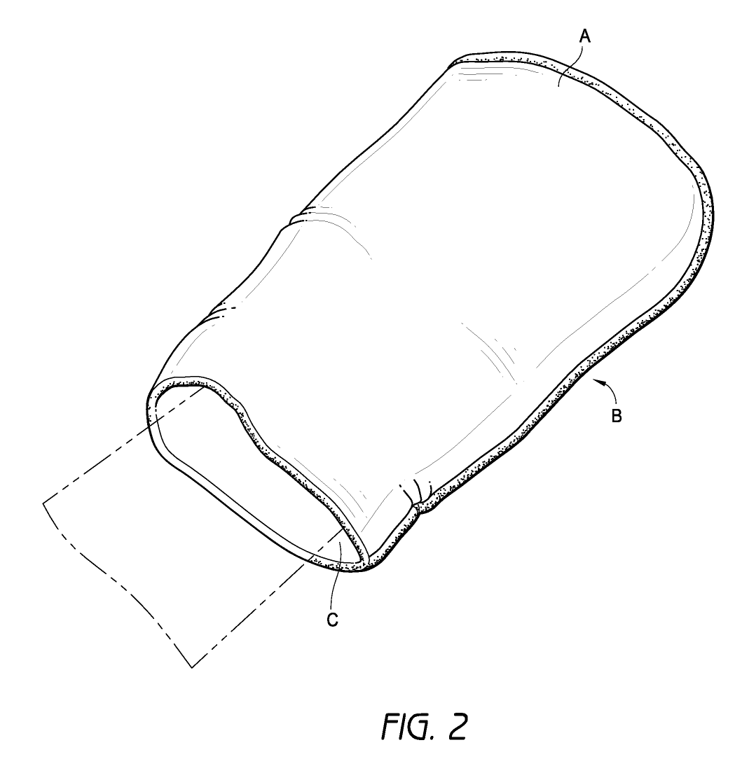

In another embodiment, the mitten can have a depth (FIG. 2, C) to cover half the arm. In a preferred embodiment, the mitten can be plastic on the outside (FIG. 2, A) so that when alkyl cyanoacrylate monomer is applied to either the wound or preferably to the mitten, which is pressed against a trauma wound, the plastic adheres to the wound after polymerization of the alkyl cyanoacrylate monomer. This seals the wound to minimize further bleeding or oozing of bodily fluids. In a further preferred embodiment, the top and bottom half of the mitten can be separated from each other after adhering to the wound to leave only the half of the mitten sealed to the wound on the patient. For example, the mitten can have a perforated edge (FIG. 2, B) that can be torn to leave the half of the mitten adhered to and around the wound in place. In some aspects, the half of the mitten sealed over the wound can be left intact until the patient is moved to treatment facilities where it can be removed, for example, by debridement.

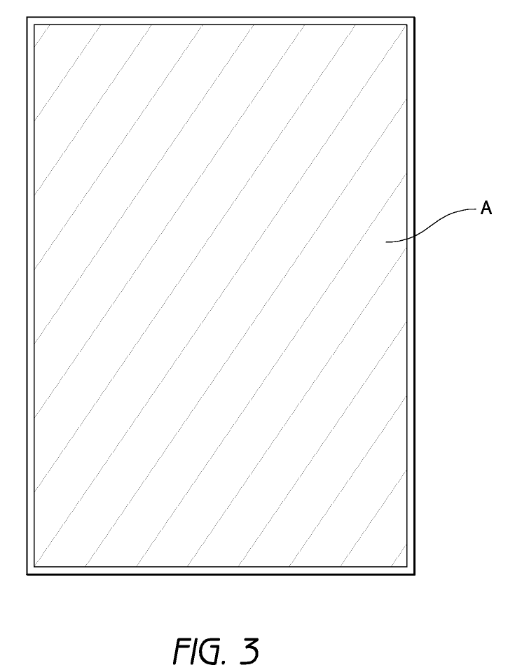

In some aspects, the injection of an alkyl cyanoacrylate into a puncture type trauma wound can slow or stop seepage of body fluids in addition to slowing or stopping bleeding. Further, if appropriate the wound can be sprayed with alkyl cyanoacrylate monomer and a plastic or other wound dressing (FIG. 3) can be pressed onto the wound holding in place for a long as needed to substantially or completely seal the wound upon polymerization of the alkyl cyanoacrylate monomer. Alternatively, and more preferably, the alkyl cyanoacrylate monomer can be applied to the dressing, which is then applied to the wound site. In a preferred embodiment, the wound dressing can adhere to and seal around the puncture type trauma wound.

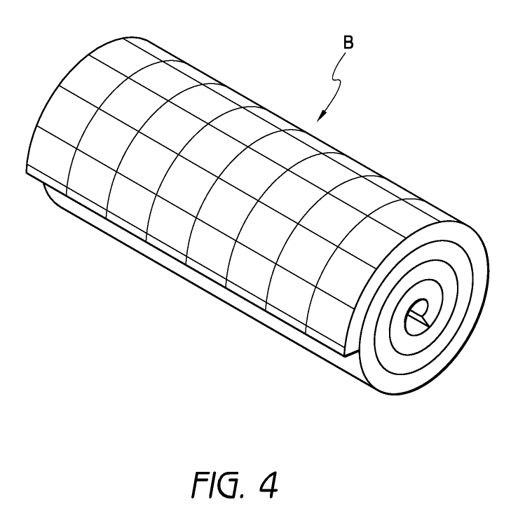

In some embodiments, the plastic wound dressing can be 4 inches by 4 inches, 5 inches by 5 inches, 4 inches by 10 inches, 10 cm by 10 cm, 10 cm by 25 cm, and the like, FIG. 2. The plastic wound dressing can be plastic on one side (FIG. 3, A) and on the other side can have a surface that does not stick to plastic film (FIG. 4, B). In some embodiments, the plastic wound dressing can be stored flat in sterile packets. In some aspects, the plastic wound dressing can be stored as a rolled tube, FIG. 4. In a preferred embodiment, the plastic wound dressing can be plastic on one side and on the other side can have a surface that does not stick to plastic film (FIG. 4, B). This can facilitate rolling the dressing up without problems with sticking to the wound. In some embodiments the side of the dressing that contacts the wound can be an impervious layer, such as a plastic or other polymer. In other embodiments, a fibrous, porous, or other type of material that can be impregnated with a cyanoacrylate is interposed between the plastic film and the wound. This cyanoacrylate-holding material can be pre-impregnated with cyanoacrylate, or it the cyanoacrylate can be applied to the material or the wound at the point of use.

In some embodiments, the time to wound sealing can be from about 1 second to about 120 seconds. In a preferred embodiment, the time to wound sealing can be from about 1 second to about 60 seconds. In a further preferred embodiment, the time to wound sealing can be from about 4 seconds to about 20 seconds.

Some embodiments relate to a method for treating and/or protecting an open type trauma wound. In some aspects, the open type trauma wound can be a severe trauma wound; for example, gunshot wounds, improvised explosive device (IED) wounds, mortar attack wounds, missile attack wounds, rocket propelled grenade (RPG) wounds, grenade wounds, bomb wounds, land mind wounds, and any military or improvised ordinance wounds.

In some embodiments, the wound or dressing can be sprayed with alkyl cyanoacrylate monomer (or the cyanoacrylate can be otherwise applied, such as through a sponge or other applicator) and the dressing can be immediately pressed onto the wound, where it is held in place for a long as needed to substantially or completely seal the wound upon polymerization of the alkyl cyanoacrylate monomer. In some aspects, the mitten or plastic film can be bound to the skin, forming a barrier to prevent further bleeding or oozing of bodily fluids.

One preferred method of the present invention involves coapplication of the cyanoacrylate field dressing product disclosed herein and a hemostatic agent. Preferred hemostatic agents include granularized chitosan material, such as that sold under the trademark CELOX by Medtrade Biopolymers, Inc, Seattle, Wash., and the chitosan bandages sold under the trademark HEMCON by Hemcon Medical Technologies, Inc., Portland, Oreg. One particularly preferred embodiment includes the use of a hemostatic agent in the dressing itself Preferred hemostatics include fibrin, diatomaceous earth, regenerated or otherwise modified cellulose, and chitosan.

In some embodiments, the alkyl cyanoacrylate monomer can be dispensed as a spray. For example, the alkyl cyanoacrylate monomer can be sprayed from a pump spray bottle. In some embodiments, the alkyl cyanoacrylate monomer can be dispensed as an aerosol from a spray bottle containing a propellant. For example, alkyl cyanoacrylate monomer can be dispensed from a sealed pressurized container, the alkyl cyanoacrylate monomer can be released in a fine spray mist when depressing a valve button located on the bottle. The aerosol spray bottle and the pump spray bottle can advantageously be impact and explosion resistant. Due to the polymerization properties of alkyl cyanoacrylate monomers precautions can be taken to prevent the spray bottle nozzle from clogging, such as by preventing monomer from entering the spray mechanism until use. Alternatively, the cyanoacrylate can be packaged in a dispenser that includes a sponge or other porous material for applying the material to a dressing or a wound. One example is a squeeze bottle with a sponge applicator on one end. The squeeze bottle can include a frangible or removable seal or other barrier to prevent the cyanoacrylate from penetrating the sponge until it is ready for use.

### Cyanoacrylate Compositions

Various known and new cyanoacrylates can be used in performing the methods disclosed herein. It is preferred that the alkyl cyanoacrylate have an alkyl chain of 3 or more carbon residues, such as propyl, butyl, pentyl, hexyl, heptyl, or octyl cyanoacrylate. The alkyl groups can be straight chain or branched, and can be attached at the 1-carbon, the 2-carbon, or at other positions.

Although the disclosure herein uses the term “monomer” to refer to the polymerizable material, this terminology should be read sufficiently broadly to encompass the use of partially polymerized cyanoacrylates, including (for example) oligomers that are further polymerizable.

As used herein the term “alkyl cyanoacrylate” refers to an adhesive compound or mixture of compounds based on cyanoacrylate monomers of formula I:

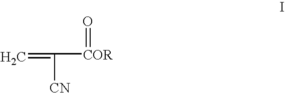

where R is selected from the group consisting of alkyl of one to sixteen carbon atoms. Partial polymers (i.e., oligomers) of such cyanoacrylates are also encompassed within this definition. Preferred R alkyl group are from 4 to 8 carbon atoms and include, by way of example, methyl, ethyl, n-butyl, isobutyl, pentyl, n-hexyl, 2-hexyl, n-heptyl, 2-heptyl, n-octyl and 2-octyl. More preferably, R is 2-hexyl, isobutyl, 2-heptyl and 2-octyl and most preferably, R is 2-hexyl.

As used herein the term “biocompatible plasticizer” refers to any material which is soluble or dispersible in alkyl cyanoacrylate, which increases the flexibility of the resulting polymer coating on the skin surface, and which is compatible with the skin as measured by the lack of skin irritation. Suitable plasticizers are well known in the art and include those disclosed in U.S. Pat. Nos. 2,784,127 and 4,444,933 the disclosures of both of which are incorporated herein by reference in their entirety. Specific stabilizers include, by way of example only, acetyl tri-n-butyl citrate, butyl benzyl phthalate, dibutyl phthalate, diethyl phthalate, dimethyl phthalate, dioctylphthalate, n-butyryl tri-n-hexyl citrate, benzoate esters of di- and poly-hydroxy branched aliphatic compounds, tri(p-cresyl) phosphate, and the like. The particular plasticizer employed is not critical provided that it does not produce skin irritation as measured by well known assays such as primary skin-irritation (rabbit). Some preferred plasticizers for use in this invention include dialkyl phthalates independently having from 1 to 10 carbon atoms in each alkyl group. A particularly preferred plasticizer is dioctylphthalate.

As used herein the term “alkyl esterified fatty acid” means a fatty acid derivatized to form an ester functional group with a alkyl moiety, such as ethyl myristate. These compounds are formed with an alkyl moiety, such as methyl, ethyl, propyl, butyl, pentyl, hexyl, heptyl, and octyl; and carboxylic acids with alkyl side chains ranging from 1 carbon, i.e., acetic acid, through to and including 17 carbons atoms in length, such as, proprionic, butyric, isobutyric, valeric, isovaleric, pivalic, lauric, myristic, palmitic and stearic acids.

As used herein the term “opacificant agent” is compound or composition which selectively absorbs or deflects radiation making the material visible under x-ray, or any like imaging technique. Typically such agents include, iodinated oils, and brominated oils, as well as commercially available compositions, such as Pantopaque, Lipiodol and Ethiodol. These commercially available compositions acts as opacificant agents, and also dilute the amount of liquid monomer thereby slowing the rate of polymerization. In addition certain metals, such as, gold, platinum, tantalum, titanium, tungsten and barium sulfate and the like, have properties enabling them to act as opacificant agents, and their use with the present cyanoacrylate compositions is contemplated.

As used herein the term “wound” refers to wounds wherein the skin is torn, cut or punctured. Open wounds can be classified into a number of different types, according to the object that caused the wound. The types of open wound that can be treated with the present invention can include but are not limited to:
Incisions or incised wounds—caused by a clean, sharp-edged object such as a knife, a razor or a glass splinter;
Lacerations—Irregular wounds caused by a blunt impact to soft tissue which lies over hard tissue (e.g. laceration of the skin covering the skull) or tearing of skin and other tissues;
Abrasions (grazes)—a superficial wound in which the topmost layers of the skin (the epidermis) are scraped off, often caused by a sliding fall onto a rough surface;
Puncture wounds—caused by an object puncturing the skin, such as a nail, knife, or bullet.
Penetration wounds—caused by an object such as a knife entering the body;
Gunshot wounds—caused by a bullet or similar projectile driving into or through the body. There may be two wounds, one at the site of entry and one at the site of exit;
Improvised explosive device (IED) wounds—caused by an object from a IED or propelled by a IED driving into or through the body. There may be two wounds, one at the site of entry and one at the site of exit;
Mortar attack wounds—caused by an object from a mortar or propelled by a mortar driving into or through the body. There may be two wounds, one at the site of entry and one at the site of exit;
Missile attack wounds—caused by an object from a missile or propelled by a missile driving into or through the body. There may be two wounds, one at the site of entry and one at the site of exit;
Rocket propelled grenade (RPG) wounds—caused by an object from a RPG or propelled by a RPG driving into or through the body. There may be two wounds, one at the site of entry and one at the site of exit;
Grenade wounds—caused by an object from a grenade or propelled by a grenade driving into or through the body. There may be two wounds, one at the site of entry and one at the site of exit;
Bomb wounds—caused by an object from a bomb or propelled by a bomb driving into or through the body. There may be two wounds, one at the site of entry and one at the site of exit;
Land mind wounds—caused by an object from a land mind or propelled by a land mind driving into or through the body. There may be two wounds, one at the site of entry and one at the site of exit;
Any military or improvised ordinance wounds—caused by an object from any military or improvised ordinance or propelled by any military or improvised ordinance driving into or through the body. There may be two wounds, one at the site of entry and one at the site of exit.

As used herein the term “stability” refers to the ability of a monomer component to resist degradation or polymerization after preparation but prior to use.

As used herein the term “inhibitor agent” refers to an agent which stabilizes a monomer composition by inhibiting polymerization. Within the context of the current invention, this term refers to agents that stabilize and inhibit polymerization by various mechanisms. By altering the amounts of one or more inhibitor agents, the rate of polymerization can be controlled. Inhibitor agents have different modes of activity, for example, hydroquinone acts primarily to inhibit high energy free radicals; p-methoxyphenol acts primarily to inhibit low energy free radicals; and phosphoric acid influences the rate of anionic polymerization.

Some suitable cyanoacrylates include substantially pure alkyl cyanoacrylate monomer or oligomer. For example, the alkyl cyanoacrylate monomer can be methyl cyanoacrylate, n-butyl cyanoacrylate, isobutyl cyanoacrylate, n-hexyl cyanoacrylate, 2-hexyl cyanoacrylate or 2-octyl cyanoacrylate, purified to about 95% purity or better. In a preferred embodiment, methyl cyanoacrylate, n-butyl cyanoacrylate, isobutyl cyanoacrylate, n-hexyl cyanoacrylate, 2-hexyl cyanoacrylate or 2-octyl cyanoacrylate can be purified to about 97% purity or better. In a more preferred embodiment methyl cyanoacrylate, n-butyl cyanoacrylate, isobutyl cyanoacrylate, n-hexyl cyanoacrylate, 2-hexyl cyanoacrylate or 2-octyl cyanoacrylate can be purified to about 98% purity or better. In a most preferred embodiment methyl cyanoacrylate, n-butyl cyanoacrylate, isobutyl cyanoacrylate, n-hexyl cyanoacrylate, 2-hexyl cyanoacrylate or 2-octyl cyanoacrylate can be purified to about 99% purity or better. In some aspects the alkyl cyanoacrylate monomer can be isolated in its crystalline form.

The stability of formulations made from alkyl cyanoacrylate monomers can be related to the purity of the monomer that is used. These properties can include but are not limited to rate of polymerization, and stability of the monomer during storage. An advantage of substantially pure alkyl cyanoacrylates can be that compositions incorporating substantially pure alkyl cyanoacrylates can require smaller amounts of additives, e.g., inhibitors, stabilizers and the like, to obtain a desired result that would otherwise have required greater amounts of the same additive. A benefit of this advantage can be in cost savings from being able to use less material. Another benefit can be that the composition will quantitatively have lower amounts of additives. This can be a desirable outcome for any composition that is subject to regulatory approval by the U.S. Food and Drug Administration, or like agency, prior to marketing. Of significant importance is the ability of the product to form a shelf-stable formulation.

Tissue toxicity was a problem for the early cyanoacrylate adhesives. As a general proposition, the longer chain alkyl cyanoacrylates have less toxicity. The toxicity of the other ingredients, including any stabilizers, polymerization inhibitors, plasticizers, and contrast agents, for example, should also be considered.

In some embodiments, the monomer component can be comprised of one or more alkyl cyanoacrylate monomers, and at least one inhibitor. In a preferred embodiment the monomer component is 2-hexyl cyanoacrylate and an inhibitor. In an especially preferred embodiment the monomer component can be comprised of 2-hexyl cyanoacrylate, and more than one inhibitor. For example, the inhibitors can be hydroquinone, p-methoxyphenol and phosphoric acid. Viscosity modification is also desirable, and sprayable compositions will generally have a lower viscosity than spreadable compositions. Viscosity modification can be achieved, for example, by solubilizing a small amount of polymerized cyanoacrylate in the monomeric cyanoacrylate, or by partially polymerizing or oligomerizing the cyanoacrylate monomer through treatment, e.g., with ultraviolet light until the desired viscosity is achieved.

One suitable polymerizable cyanoacrylate material that can be used in this invention is a 2-hexyl cyanoacrylate made by Valor Medical, Inc., San Diego, Calif., under the trademark NEUCRYLATE. Other suitable materials are disclosed, for example, in U.S. patent application publication Nos. 20050197421 and 20050196376, the entire contents of which are incorporated by this reference. One preferred material is a single vial 2-hexyl cyanoacrylate disclosed in U.S. Provisional Patent Application No. 60/987,349, which is incorporated herein by reference in its entirety. NEUCRYLATE and the single vial formulation referenced here are essentially non-toxic, have enhanced flexibility, polymerize rapidly (in a matter of seconds) after contact with body fluids, and are compatible and non-interfering with hemostatic agents such as the granularized chitosan material sold under the trademark CELOX by Medtrade Biopolymers, Inc, Seattle, Wash.

It has been discovered that 2-hexyl cyanoacrylate compositions have antibacterial properties, both in their unpolymerized and polymerized form. This makes them particularly suitable for use in wound treatments. Those same compositions have minimal toxicity, making them suitable for both topical and internal use.

In some embodiments, the monomer component can be comprised of two or more different alkyl cyanoacrylate monomers, and at least one inhibitor. In a preferred aspect of the present embodiment, the monomer component can be comprised of methyl cyanoacrylate, n-hexyl cyanoacrylate and at least one inhibitor. In one exemplary embodiment, the monomer component can be comprised of methyl cyanoacrylate, n-hexyl cyanoacrylate and at least three inhibitors, for example, the inhibitors can be hydroquinone, p-methoxyphenol and acetic acid.

In some embodiments, particularly when the cyanoacrylate is injected into a puncture wound, it may be desirable to include a contrast agent to permit visualization of the wound track. Suitable opacificants can include gold particles and iodinated materials.

Some embodiments can provide a substantially pure alkyl cyanoacrylate monomer. For example, the alkyl cyanoacrylate monomer can be methyl cyanoacrylate, n-butyl cyanoacrylate, isobutyl cyanoacrylate, n-hexyl cyanoacrylate, 2-hexyl cyanoacrylate or 2-octyl cyanoacrylate, purified to about 95% purity or better. In a preferred embodiment, methyl cyanoacrylate, n-butyl cyanoacrylate, isobutyl cyanoacrylate, n-hexyl cyanoacrylate, 2-hexyl cyanoacrylate or 2-octyl cyanoacrylate can be purified to about 97% purity or better. In a more preferred embodiment methyl cyanoacrylate, n-butyl cyanoacrylate, isobutyl cyanoacrylate, n-hexyl cyanoacrylate, 2-hexyl cyanoacrylate or 2-octyl cyanoacrylate can be purified to about 98% purity or better. In a most preferred embodiment methyl cyanoacrylate, n-butyl cyanoacrylate, isobutyl cyanoacrylate, n-hexyl cyanoacrylate, 2-hexyl cyanoacrylate or 2-octyl cyanoacrylate can be purified to about 99% purity or better. In some aspects the alkyl cyanoacrylate monomer can be isolated in its crystalline form.

In some embodiments, the monomer component of the present invention can be comprised of at least one alkyl cyanoacrylate and at least one inhibitor agent. Typical inhibitors appropriate for cyanoacrylates can be, for example, hydroquinone, p-methoxyphenol, pure phosphoric acid, and alkyl carboxylic acids, where the alkyl moiety can range from 1 carbon, e.g., acetic acid, through to 15 and 17 carbons atoms in length, i.e., palmitic and stearic acids, respectively; and phosphoric acid at varying percentage solutions. In a preferred aspect, hydroquinone, p-methoxyphenol, acetic acid and phosphoric acid can be used, individually or in combination.

Different inhibitors have different physical characteristics and thereby functions to alter the final properties of the composition. For example, hydroquinone can be used as an inhibitor for high energy free radicals; p-methoxyphenol can be used as an inhibitor for low energy free radicals; and phosphoric acid can act to control or inhibit anionic polymerization and the rate of such polymerization.

The quantity of inhibitors used can be measured in terms of parts per million of alkyl cyanoacrylate. For example, for 2-hexyl cyanoacrylate, hydroquinone can be in the range of about 50 to about 150 parts per million (PPM), p-methoxyphenol in the range of about 50 to about 150 PPM, and phosphoric acid in the range of about 125 to about 375 PPM. In a preferred embodiment, hydroquinone can be in the range of about 75 to about 125 PPM, p-methoxyphenol in the range of about 75 to about 125 PPM, and phosphoric acid in the range of about 187.5 to about 312.5 PPM. In a most preferred embodiment, hydroquinone can be in the range of about 95 to about 105 PPM, p-methoxyphenol in the range of about 95 to about 105 PPM, and phosphoric acid in the range of about 200 to about 300 PPM. Similarly, for a monomer component comprising of 90% n-hexyl cyanoacrylate and 10% methyl cyanoacrylate, hydroquinone can be in the range of about 50 to about 150 PPM, p-methoxyphenol is in the range of about 50 to about 150 PPM, and acetic acid is in the range of about 50 to about 500 PPM. In a more preferred embodiment, hydroquinone can be in the range of about 75 to about 125 PPM, p-methoxyphenol in the range of about 75 to about 125 PPM and acetic acid in the range of about 100 to about 300 PPM. In a most preferred embodiment, hydroquinone can be in the range of about 95 to about 105 PPM, p-methoxyphenol in the range of about 95 to about 105 PPM, and acetic acid in the range of about 150 to about 250 PPM.

Although the foregoing discussion touches on many particular aspects and features of the invention, the scope of the exclusive rights should not be limited to particular embodiments disclosed above, but instead should be measured by the full, lawful, valid scope of the claims that follow:

What is claimed is:

1. A method for treating a puncture wound, comprising:
providing a biocompatible alkyl cyanoacrylate composition that can polymerize upon contact with body fluid; and
introducing the composition into a puncture wound to seal the wound and reduce bleeding.

2. The method of claim 1, wherein the alkyl cyanoacrylate has an alkyl moiety of from 3-8 carbon atoms.

3. The method of claim 1, wherein the alkyl moiety is n-hexyl.

4. The method of claim 1, wherein the alkyl moiety is 2-hexyl.

5. The method of claim 2, wherein the alkyl cyanoacrylate is stored in a single vial formulation prior to use.

6. A method for treating an open trauma wound, comprising:
providing a wound dressing;
applying the wound dressing and a polymerizable alkyl cyanoacrylate to the wound; and
maintaining the wound dressing on the wound until the alkyl cyanoacrylate polymerizes, thereby sealing the wound and inhibiting blood loss from the wound.

7. The method of claim 6, wherein the alkyl cyanoacrylate has an alkyl moiety of from 3-8 carbon atoms.

8. The method of claim 6, wherein the alkyl moiety is n-hexyl.

9. The method of claim 6, wherein the alkyl moiety is 2-hexyl.

10. The method of claim 9, wherein the alkyl cyanoacrylate is bactericidal or bacteristatic.

11. The method of claim 6, wherein the wound dressing comprises a substantially impermeable layer.

12. The method of claim 6, wherein the wound dressing comprises a polymer film.

13. The method of claim 6, wherein the wound dressing has an opening to permit insertion of a user's hand.

14. The method of claim 13, wherein the wound dressing is in the form of a mitten.

15. The method of claim 13, wherein the wound dressing comprises top and bottom layers of polymer film, in which the applying step further comprises inserting a user's hand between the layers and pressing the bottom layer against the wound until the bottom layer adheres to the wound.

16. The method of claim 6, further comprising coating either the wound or the dressing with the cyanoacrylate prior to applying the wound dressing.

17. The method of claim 16, wherein the wound dressing is coated with the cyanoacrylate.

18. The method of claim 16, wherein the coating step comprises spraying the cyanoacrylate.

19. The method of claim 6, further comprising applying a hemostatic agent to the wound.

20. The method of claim 19, wherein the hemostatic agent comprises chitosan.

21. The method of claim 19, wherein the dressing comprises said hemostatic agent.

22. The method of claim 19, wherein the hemostatic agent is granular.

23. The method of claim 6, wherein the wound dressing is self-applied by a wound victim.
